(19) United States
(12) Patent Application Publication
(10) Pub. No.: US 2021/0322704 A1
(43) Pub. Date: Oct. 21, 2021

(54) Methods of respiratory support and related apparatus

(71) Applicant: Edward D. Lin, (US)

(72) Inventor: Edward D. LIN, Osprey, FL (US)

(21) Appl. No.: 17/141,138

(22) Filed: Jan. 4, 2021

Related U.S. Application Data
(63) Continuation-in-part of application No. 16/851,405, filed on Apr. 17, 2020, now abandoned.

(51) Int. Cl. A61M 16/06; A61M 16/20; A61M 16/10

(52) U.S. Cl. CPC A61M 16/0672; A61M 16/201; A61M 2205/75; A61M 2205/50; A61M 16/101

(57) ABSTRACT

Methods of respiratory support and related respiratory support apparatus are disclosed. The methods may include a first step of communicating only a respiratory gas from a gas source into a regulator chamber of a regulator during inhaling by a user and a second step of communicating ambient air from an ambient environment into the regulator chamber during inhaling by the user. The first step and the second step are sequenced thereby communicating only the respiratory gas into lungs of the user during the first step and communicating the ambient air into an anatomical dead space of the user during the second step.

### CROSS-REFERENCE TO RELATED APPLICATIONS

This application is a continuation-in-part of U.S. patent application Ser. No. 16/851,405 filed 17 Apr. 2020, which is hereby incorporated by reference in its entirety herein.

### BACKGROUND OF THE INVENTION

### Field

This disclosure relates to apparatus and related methods for supplying breathable gas to a user, and more specifically, to apparatus and related methods for supporting respiration in users suffering from respiratory deficiencies.

### Background

As an example of the need for respiratory support, SARS-CoV-2, which causes COVID-19, has proven to be a highly infectious, virulent coronavirus that may have a mortality rate higher than influenza. While elderly patients with underlying medical conditions are more at risk, everyone is vulnerable. Infants as well as healthy adults have succumbed to this pathogen that has a predilection for the respiratory tract. The hallmark of COVID-19 lethality is severe respiratory failure that may occur quickly. Even patients seemingly asymptomatic may have shockingly low oxygen saturation. For example, a patient whose oxygen saturation drops from near 99% to 85% may be deemed at imminent risk for cardiopulmonary arrest. And yet seemingly fit young COVID-19 patients without shortness of breath have been found with oxygen saturation in the 70% range. Therefore, these COVID-19 patients may have little to no margin of safety and should receive aggressive oxygen support early on.

The virus attacks not only the lung tissues, but also the heart, liver and endothelium that lining of blood vessels resulting in complications. In the lungs, plasma seeps out of the vascular tree into the alveolar space further impeding oxygen exchange. This subset of patients spiral into respiratory failure that is resistant to ventilatory support and approximately 52% to 85% die.

COVID-19 patients in respiratory failure may require prolonged ventilator support. Ventilators are complex machines that require deep medical knowledge to operate and are typically used in a hospital ICU setting. A ventilator may require an oxygen supply of about 200 liters per minute (LPM) to about 250 LPM. While ventilators may save lives, ventilators may cause grave complications such as perforated lungs and hemodynamic collapse. In an acute setting, sedatives and paralytic drugs may be required to keep the patients from fighting the ventilator due to the intubation. A majority of COVID-19 ventilated patients die on the ventilator from refractory hypoxia, as their friable damaged lungs are poorly able to deal with the trauma of forced ventilation. In addition to COVID-19 patients, those with serious end-organ disease such as congestive heart failure or chronic obstructive pulmonary disease also need effective oxygenation to protect and sustain a meaningful quality of life.

A high frequency nasal cannula (HFNC) may be an alternative to a ventilator. The HFNC relies on spontaneous breathing but an oxygen supply of about 60-80 LPM. The HFNC is typically used in a hospital ICU setting.

Achievement of near 100% oxygenation at the alveolar level is normally assumed to require either a ventilator or a HFNC. Yet it is sometimes difficult to get even a hospital bed, let alone an ICU bed, to receive such treatment, which may result in additional suffering and loss of life.

On the other end of the spectrum, users such as professional athletes, mountain climbers and military operatives due for deployment in high altitude locations may train in a relatively low oxygen environment in order to induce erythropoietin production. Erythropoietin stimulates the bone marrow to produce more red blood cells. The resulting rise in red cells increases the oxygen-carrying capacity of the blood but the process takes 1-2 months. For example, special military training facilities simulate such relative hypoxia by exposing soldiers to oxygen concentration in the 10% to 17% range for up to 1.5 hours multiple times a day (Intermittent/Interval Hypoxic Treatment (IHT). Such acclimatization usually requires troop movement to specially equipped facilities and deployment may not always be matched to training time. Sudden deployment may mean that soldiers are not optimally acclimatized.

High altitude illnesses include acute mountain sickness (AMS), high altitude cerebral edema (HACE) and high-altitude pulmonary edema (HAPE) may occur. When soldiers develop AMS, for example, they suffer not only from low ambient oxygen, but also a variable degree of HAPE from low ambient pressure which insufficiently counters the outward hydrostatic pressure of the vascular tree, resulting in seepage of plasma into the alveolar space. This further compromises oxygen exchange in the face of already low ambient oxygen. The affected soldiers normally have to be returned to a lower altitude to recover, risking compromise to mission readiness.

Accordingly, there is a need for improved methods of respiratory support as well as related apparatus. Such improved methods of respiratory support and related apparatus, for example, may be used for treatment of hypoxia that may avert the need for ventilator support, and may be adapted for hypoxia training for specialized purposes as well as treatment for high altitude sickness.

### BRIEF SUMMARY OF THE INVENTION

These and other needs and disadvantages may be overcome by the methods and related apparatus disclosed herein. Additional improvements and advantages may be recognized by those of ordinary skill in the art upon study of the present disclosure.

In various aspects, the methods may include the step of communicating only a respiratory gas from a gas source into a regulator chamber of a regulator during inhaling by a user, the regulator chamber being in communication with a facemask chamber of a facemask adapted for securement over an inspiratory intake of the user. In various aspects, the methods may include the step of communicating ambient air from an ambient environment into the regulator chamber during inhaling by the user following the step of communicating only a respiratory gas from a gas source and from a bag reservoir into a regulator chamber. The methods may include sequencing the step of communicating only a respiratory gas from a gas source into a regulator chamber with the step of communicating ambient air from an ambient environment into the regulator chamber thereby communicating only the respiratory gas into lungs of the user and communicating the ambient air into an anatomical dead space of the user, in various aspects.

In various aspects, the step of communicating only a respiratory gas from a gas source into a regulator chamber of a regulator during inhaling by a user may include opening a check valve as a user is inhaling. The check valve may be opened by the user inhaling. In various aspects, the step of communicating ambient air from an ambient environment into the regulator chamber during inhaling by the user may include opening an anti-asphyxiation valve as the user is inhaling. The anti-asphyxiation valve may be opened by the user inhaling. The opening of the check valve as the user is inhaling may be sequenced with the opening of the anti-asphyxiation valve as the user is inhaling thereby communicating only the respiratory gas into lungs of the user and communicating the ambient air into an anatomical dead space of the user. Ambient air may be communicated into a facemask chamber of the facemask and the regulator chamber of the regulator.

In various aspects, for example, about 350 ml of respiratory gas are communicated into the regulator chamber during inhaling by the user followed by communicating about 150 ml of ambient air into the regulator chamber during inhaling by the user. In various aspects, a bag defining a bag reservoir may be in communication with the regulator, and depleting of respiratory gas within the bag reservoir may initiate opening of the anti-asphyxiation valve. The methods may include sizing a volume of the bag reservoir to initiate the opening of the anti-asphyxiation valve concurrent with complete filling of the lungs with respiratory gas. The volume of the bag reservoir may vary depending upon the anatomy of the user.

The respiratory gas may comprise oxygen at a concentration greater than that of ambient air. For example, in various aspects, the respiratory gas may be provided by an oxygen concentrator that may supply about 85% to about 94% oxygen at a continuous flow of 5 L/min (LPM). In various aspects, the respiratory gas may be provided at a continuous flow rate of about 5 LPM to about 10 LPM.

This summary is presented to provide a basic understanding of some aspects of the apparatus and methods disclosed herein as a prelude to the detailed description that follows below. Accordingly, this summary is not intended to identify key elements of the apparatus and methods disclosed herein or to delineate the scope thereof.

### BRIEF DESCRIPTION OF THE DRAWINGS

FIG. 1A illustrates by perspective view an exemplary implementation of a respiratory support apparatus;

FIG. 1B illustrates by another perspective view the exemplary respiratory support apparatus of FIG. 1A;

FIG. 2 illustrates by perspective view portions of the exemplary respiratory support apparatus of FIG. 1A;

FIG. 3 illustrates by cut-away perspective view portions of the exemplary respiratory support apparatus of FIG. 1A;

FIG. 4A illustrates by cut-away side view portions of the exemplary respiratory support apparatus of FIG. 1A including a check valve in a closed position;

FIG. 4B illustrates by side cut-away view portions of the exemplary respiratory support apparatus of FIG. 1A including the check valve of FIG. 4A in an open position;

FIG. 5A illustrates by top view portions of the exemplary respiratory support apparatus of FIG. 1A including an anti-asphyxiation valve;

FIG. 5B illustrates by side cut-away view portions of the exemplary respiratory support apparatus of FIG. 1A including the anti-asphyxiation valve of FIG. 5A in a closed position;

FIG. 5C illustrates by side cut-away view portions of the exemplary respiratory support apparatus of FIG. 1A including the anti-asphyxiation valve of FIG. 5A in an open position;

FIG. 6A illustrates by cut-away perspective view portions of the exemplary respiratory support apparatus of FIG. 1A including an exemplary implementation of a filter disposed within an anti-pathogen module;

FIG. 6B illustrates by side cross-sectional view the portions of the exemplary respiratory support apparatus of FIG. 1A that are illustrated in FIG. 6A;

FIG. 6C illustrates by cross-section side view portions of the exemplary respiratory support apparatus of FIG. 1A including another exemplary implementation of a filter disposed within the exemplary anti-pathogen module of FIG. 6A;

FIG. 7 illustrates by schematic diagram portions of the exemplary respiratory support apparatus of FIG. 1A;

FIG. 8A illustrates by schematic diagram the exemplary respiratory support apparatus of FIG. 1A in a first operational state;

FIG. 8B illustrates by schematic diagram the exemplary respiratory support apparatus of FIG. 1A in a second operational state;

FIG. 8C illustrates by schematic diagram the exemplary respiratory support apparatus of FIG. 1A in a third operational state;

FIG. 9 illustrates by cut-away perspective view a second exemplary implementation of a respiratory support apparatus;

FIG. 10 illustrates by cut-away perspective view portions of the second exemplary implementation of a respiratory support apparatus of FIG. 9;

FIG. 11 illustrates by exploded perspective view portions of the second exemplary implementation of a respiratory support apparatus of FIG. 9;

FIG. 12 illustrates by process flow chart exemplary operations of the exemplary respiratory support apparatus of FIG. 1A and the exemplary respiratory support apparatus of FIG. 9; and,

FIG. 13 illustrates a facemask in accordance with the exemplary respiratory support apparatus of FIG. 1A or the exemplary respiratory support apparatus of FIG. 9 secured to a user along with certain exemplary anatomical features of the user.

The Figures are exemplary only, and the implementations illustrated therein are selected to facilitate explanation. The number, position, relationship and dimensions of the elements shown in the Figures to form the various implementations described herein, as well as dimensions and dimensional proportions to conform to specific force, weight, strength, flow and similar requirements are explained herein or are understandable to a person of ordinary skill in the art upon study of this disclosure. Where used in the various Figures, the same numerals designate the same or similar elements. Furthermore, when the terms “top,” “bottom,” “right,” “left,” “forward,” “rear,” “first,” “second,” “inside,” “outside,” and similar terms are used, the terms should be understood in reference to the orientation of the implementations shown in the drawings and are utilized to facilitate description thereof. Use herein of relative terms such as generally, about, approximately, essentially, may be indicative of engineering, manufacturing, or scientific tolerances such as ±0.1%, ±1%, ±2.5%, ±5%, or other such tolerances, as would be recognized by those of ordinary skill in the art upon study of this disclosure.

### DETAILED DESCRIPTION OF THE INVENTION

A respiratory support apparatus that includes a regulator attachable onto a facemask for communication of fluid between a regulator chamber defined by the regulator and a facemask chamber defined by the facemask is disclosed herein. In certain aspects, the attachment between the facemask and the regulator is rigid. Respiratory gas is communicated into the regulator from a gas source, in various aspects. Check valves disposed within the regulator chamber control the flow of respiratory gas into the facemask chamber and the flow of outflow gas from the facemask chamber as a user breathes, in various aspects. A Positive End Expiratory Pressure valve (PEEP valve) may be optionally disposed within a pathway of the outflow gas to maintain a selected baseline pressure p_{BL} within the regulator chamber as the user exhales, in various aspects. An anti-pathogen module may be included in the respiratory support apparatus to filter or disinfect outflow gas, in various aspects. Inclusion of the antipathogen module may reduce the risk of pathogen transmission or may obviate the need for a negative pressure air ventilation system for pathogen control in a room in which the user is situated.

In various aspects, the facemask may be, for example, a standard anesthesia facemask, a resuscitation facemask, or other leak resistant facemask with or without an inflatable cushion, and the regulator may be configured to connect to a mask conduit of the facemask. When used in conjunction with an anesthesia facemask, for example, the regulator may enable the anesthesia facemask to be reused in a post anesthesia care unit (PACU) for continued oxygenation of the user post-surgery. Use of the regulator with the anesthesia facemask in PACU may reduce costs and the generation of medical waste by eliminating the need for an additional facemask for use in PACU. The regulator in combination with anesthesia facemask may deliver greater inspired oxygen concentration than may be delivered currently in PACU.

The respiratory support apparatus disclosed herein may be used for oxygen supplementation of spontaneously breathing users, in various aspects. In such uses, the respiratory support apparatus may provide a higher fraction (up to 100%) of inspired oxygen (FiO2) than nasal cannula (about 35%) while being non-invasive. Because, in various aspects, the respiratory support apparatus is non-invasive and relies on spontaneous respiration of the user, the respiratory support apparatus may provide advantages over ventilator-mediated respiration, including: [1] elimination of risk of respiratory arrest if endotracheal tube is dislodged while the user remains paralyzed and/or sedated [2] elimination of ventilator-dependency and of inability to be weaned off of mechanical ventilation, [3] no circumvention of natural air filtering and immune defenses provided by nasal turbinates, lymphoid tissue, and pharyngeal mucosa as would occur with use of an endotracheal tube, the endotracheal tube being associated with high risk of nosocomial infections; and [4] reduction of cost associated with ventilator use and ICU stay. Because the respiratory support apparatus may be single use, in various aspects, disposal following use may aid infection control.

The respirator support apparatus disclosed herein may be used in situations where a number of people occupy a confined space, and at least one person has an infectious disease, in various aspects. In the case of COVID-19, for example, infected persons may be hypoxic but asymptomatic. These hypoxic persons may continue to carry out their duties, especially when their oxygen deficit is being treated. For example, in a scientific or military mission where every person has an important task and transmission of infection could lead to mission failure, the respiratory support apparatus may provide a margin of safety for each individual and enhance the likelihood of mission success. In such aspects, oxygen may be conveyed from a liquid oxygen tank and distributed to a workstation with a pigtail hose to allow certain freedom of movement, in various aspects.

As used herein, a user is defined as a person to whom the facemask of the respiratory support apparatus is attached. In certain aspects, a healthcare provider may employ the respiratory support apparatus in treating the user, or the healthcare provider may be the user for protection against infection transmission from others. Healthcare provider may be, for example, a physician, physician's assistant, nurse, or respiratory therapist.

As used herein, the terms distal and proximal are defined from the point of view of the healthcare provider treating the user with the respiratory support apparatus. A distal portion of the respiratory support apparatus is oriented toward the user while a proximal portion of the respiratory support apparatus is oriented toward the healthcare provider. In general, a distal portion of a structure may be closest to the user (e.g. the patient) while a proximal portion of the structure may be closest to the healthcare provider treating the user.

Ambient pressure p_{amb}, as used herein, refers to the pressure in a region surrounding the respiratory support apparatus. Ambient pressure p_{amb}, for example, may refer to atmospheric pressure, hull pressure within an aircraft where the respiratory support apparatus is being utilized, or pressure maintained within a building or other structure where the respiratory support apparatus is being utilized. Ambient pressure p_{amb} may vary, for example, with elevation or weather conditions. Unless specifically stated, pressure as used herein is gauge pressure, that is, pressure relative to ambient pressure p_{amb}. Positive pressures indicate pressures greater than ambient pressure p_{amb}, and negative pressures indicate pressures less than ambient pressure p_{amb}.

A computer, as used herein, includes, a processor that may execute computer readable instructions operably received by the processor. The computer may be, for example, a single-processor computer, multiprocessor computer, multi-core computer, minicomputers, mainframe computer, supercomputer, distributed computer, personal computer, hand-held computing device, tablet, smart phone, and a virtual machine, and the computer may include several processors in networked communication with one another. The computer may include memory, screen, keyboard, mouse, storage devices, I/O devices, and so forth, in various aspects, that may be operably connected to a network. The computer may execute various operating systems (OS) such as, for example, Microsoft Windows, Linux, UNIX, MAC OS X, real time operating system (RTOS), VxWorks, INTEGRITY, Android, iOS, or a monolithic software or firmware implementation without a defined traditional operating system. Compositions of matter disclosed herein include non-transitory media that includes computer readable instructions that, when executed, cause one or more computers to function as at least a portion of the apparatus disclosed herein or to implement at least a portion of the method steps of the methods disclosed herein.

Network, as used herein, may include the Internet cloud, as well as other networks of local to global scope. Network may include, for example, data storage devices, input/output devices, routers, databases, computers including servers, mobile devices, wireless communication devices, cellular networks, optical devices, cables, and other hardware and operable software, as would be readily recognized by those of ordinary skill in the art upon study of this disclosure. Network may be wired (e.g. optical, electromagnetic), wireless (e.g. infra-red (IR), electromagnetic), or a combination of wired and wireless, and the network may conform, at least in part, to various standards, (e.g. Bluetooth®, FDDI, ARCNET, IEEE 802.11, IEEE 802.20, IEEE 802.3, IEEE 1394-1995, USB).

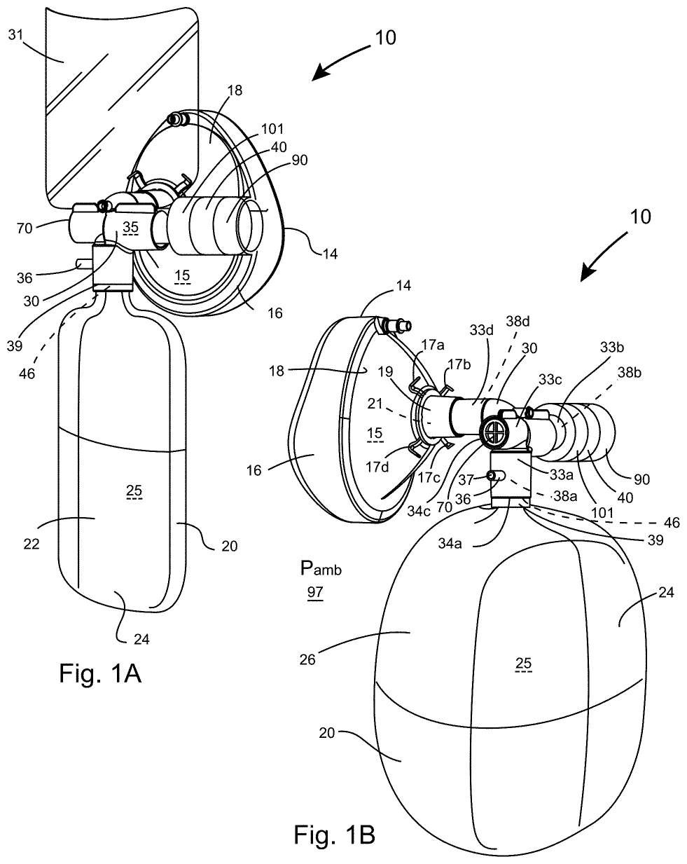

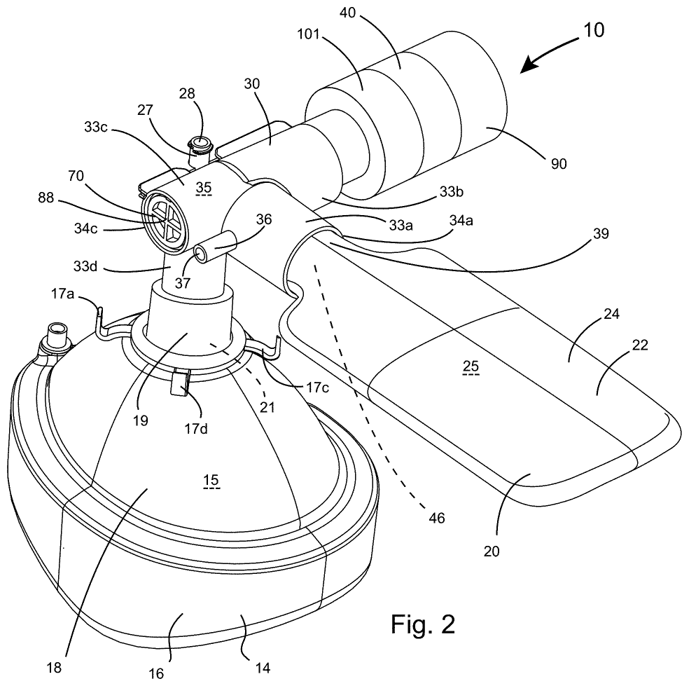

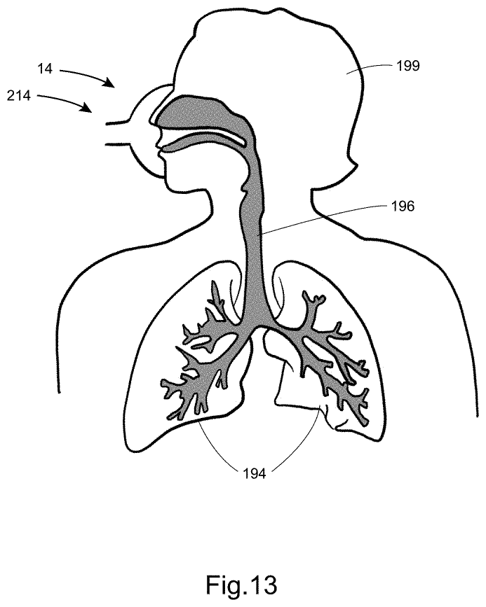

FIGS. 1A, 1B and FIG. 2 illustrate exemplary respiratory support apparatus 10 including regulator 30 pivotably secured to facemask 14. Facemask 14 includes dome 18 surrounded peripherally by cushion 16, and mask conduit 19, which defines conduit passage 21, extends forth from dome 18, as illustrated. When secured to a user, such as user 199 (see FIG. 13), by head-strap(s) (not shown) engaged with head-strap hooks 17a, 17b, 17c, 17d, facemask 14 defines facemask chamber 15 over the user's nose and mouth. Facemask 14 may be formed as an anesthesia mask, in various implementations. Dome 18 may be formed, for example, of rigid clear, polymer such as polyethylene terephthalate (PET), copolyester (such as Eastman Tritan®) or polycarbonate. Cushion 16 may be formed of soft polymer such as PVC or silicone. Cushion 16 may be adjustably inflatable, in various implementations. As illustrated in FIG. 1B, ambient environment 97 has ambient pressure p_{amb}.

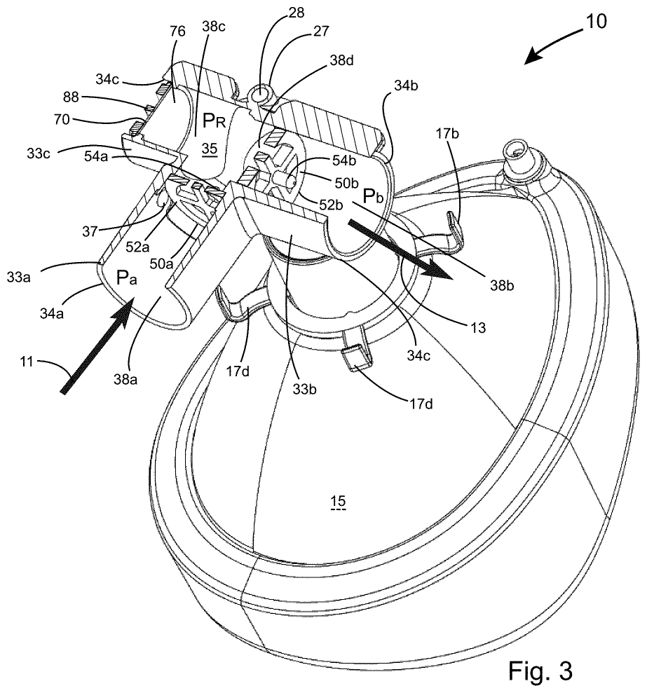

Regulator 30 includes arms 33a, 33b, 33c generally in coplanar disposition in the form of a “Y” or “T” and arm 33d generally normal to the coplanar disposition of arms 33a, 33b, 33c. Various implementations may have other relational orientations of arms 33a, 33b, 33c, 33d with respect to one another. Regulator 30 defines regulator chamber 35 and arms 33a, 33b, 33c, 33d define arm passages 38a, 38b, 38c, 38d, respectively, that communicate fluidly with regulator chamber 35, as illustrated in FIG. 3. Check valves 50a, 50b and anti-asphyxiation valve 70 are disposed within arm passages 38a, 38b, 38c, respectively, to control fluid communications with regulator chamber 35 and, thus, with facemask chamber 15, as illustrated in FIGS. 2, 3. At least portions of regulator 30 including arms 33a, 33b, 33c, 33d, at least portions of check valves 50a, 50b, and at least portions of anti-asphyxiation valve 70 may be formed of various suitable plastics, for example, by 3-D printing including other reproduction or additive technologies that may facilitate manufacture including manufacture in situ.

While the facemask chamber 15 encloses the user's mouth and nose to protect the mouth and nose, respiratory support apparatus 10 includes shield 31 that is attached to regulator 30 to form a barrier, for example, against infectious aerosol that may otherwise be directed at the user's eyes, as illustrated in FIG. 1A. Note that shield 31 is omitted from the other Figures for purposes of clarity of explanation. Shield 31 may be formed of a transparent material such as acrylic sheet. Shield 31 is optional and may be omitted, in other implementations.

As illustrated in FIG. 2, regulator 30 is rotatably secured in a fluid tight manner to mask conduit 19 by the engagement of arm 33d with mask conduit 19 to allow fluid communication between regulator chamber 35 and facemask chamber 15 via arm passage 38d of arm 33d and via conduit passage 21 of mask conduit 19. For example, arm 33d of regulator 30 may be secured to mask conduit 19 of facemask 14 by interference or compression fit according to ISO standard. Arm 33d and mask conduit 19 are rigid so that regulator 30 is rigidly and engaged with facemask 14, in this implementation. In other implementations, regulator 30 may be flexibly engaged with facemask 14. It is contemplated that regulator 30 may be disposed proximate facemask 14 to facilitate fluid communication between facemask chamber 15 and regulator chamber 35. When secured to facemask 14, regulator 30 may be pivoted about arm 33d as axis to position arms 33a, 33b, 33c in various orientations with respect to the user when facemask 14 is affixed to the user. Mask conduit 19 including conduit passage 21 may be of a standard size and standard configuration, such as those prescribed by ISO 5361:2016 standards governing anesthesia masks and ventilation equipment. Arm 33d may be sized and otherwise configured for secure engagement with mask conduit 19 having the standard size. For example, arm 33d may be sized to be insertably securably received within conduit passage 21 of mask conduit 19. Accordingly, regulator 30 is configured to fit existing facemask 14 to form portions of respiratory support apparatus 10, in various implementations.

As illustrated in FIGS. 1A, 1B, 2, bag 20 is affixed to arm end 34a of arm 33a of regulator 30, for example, by compression fit of bag conduit 39 and arm 33a to allow fluid communication between bag reservoir 25, which is defined by bag 20, and regulator chamber 35 of regulator 30. Bag 20 is appended to bag conduit 39 that defines bag conduit passage 46 through which bag reservoir 25 fluidly communicates with arm passage 38a, as illustrated. In FIG. 1A, bag 20 is illustrated in collapsed state 22, which may occur in later portions of user inhalation when respiratory gas 11 (see FIG. 3) is generally withdrawn from bag reservoir 25. In FIG. 1B, bag 20 is illustrated in expanded state 26, which may occur proximate completion of user exhalation when bag reservoir 25 is generally filled with respiratory gas 11. Bag 20 may be formed of compliant fluid-impermeable material such as polyethylene sheeting, and bag 20 may have display color 24 that enhances the visual apprehension of bag 20 to allow visual assessment of respiratory function. Display color 24 may be, for example, safety orange, safety red, safety green, or other bright neon color, pattern, or combination of color and pattern that aids a healthcare provider in perceiving bag 20 in collapsed state 22, expanded state 26, and as bag 20 transitions between collapsed state 22 and expanded state 26.

For example, the healthcare provider observes the excursion of the chest wall and times the excursion of the chest wall to estimate respiratory conditions such as tidal volume and respiratory rate of the user. If, for example, the user has COPD (chronic obstructive pulmonary disease) or is obese, the chest wall excursion may become difficult for the healthcare provider to assess and the chest wall excursion may be impossible to assess from even a short distance away. Display color 24 of bag 20 may allow the healthcare provider to assess the transitioning of bag 20 as bag transitions between collapsed state 22 and expanded state 26 thereby allowing estimation of respiratory conditions of the user. The amount of expansion and collapse, for example, allows for estimation of the tidal volume. In a ward with many users, for example, display color 24 of bag 20 may allow the healthcare provider to assess more accurately the respiratory adequacy of many users nearly simultaneously. For example, a user with rapid bag expansion—collapse (possibly indicating respiratory distress), or abnormally low bag expansion—collapse (possibly indicating respiratory depression) are users to whom prompt attention may be required.

As illustrated in FIGS. 2, 3, inflow port 36 formed as a nipple on arm 33a defines inflow passage 37. Tubing including various piping, hose(s), connector(s), and other fluid conveyances (not shown) for the conveyance of respiratory gas 11 may be received by inflow port 36 to fluidly communicate respiratory gas 11 into regulator chamber 35 via inflow passage 37. Respiratory gas 11 includes, for example, oxygen or oxygen in combination with other gas(ses), in various implementations. In certain implementations, respiratory gas 11 may have an oxygen concentration greater than that of ambient air 12, which is about 20.95% oxygen by volume. In certain implementations, respiratory gas 11 may have an oxygen concentration in a range of about 85% to about 94% oxygen.

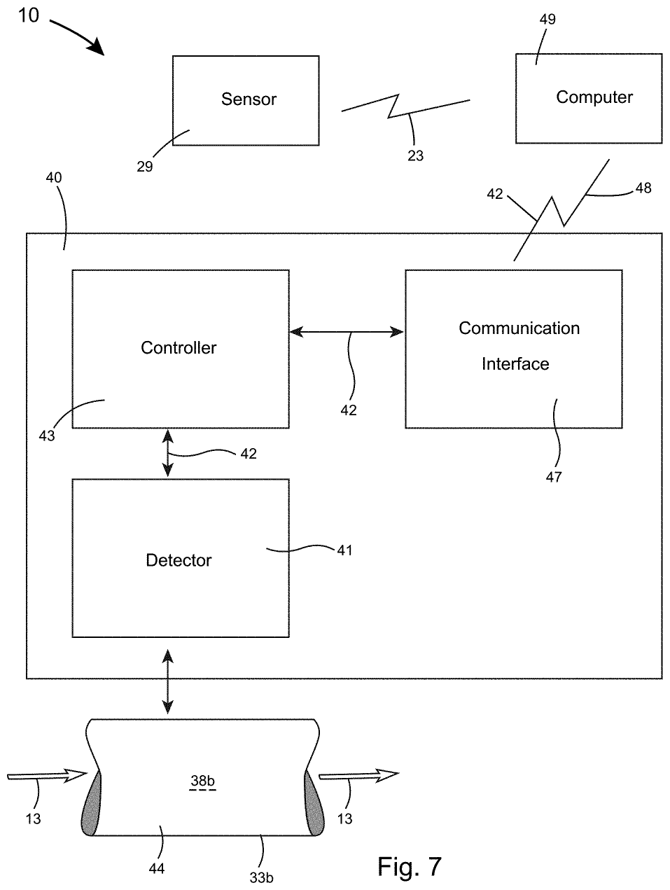

Sensor port 27 on regulator 30 defines sensor passage 28 that communicates through regulator 30 with regulator chamber 35. Sensor 29 (see FIG. 7) may be received within sensor passage 28 to detect an attribute, such as attribute 44, within regulator chamber 35, within arm passage 38d, and/or within facemask chamber 15. Attribute 44 may include, for example, EtCO2 (end tidal carbon dioxide, for monitoring adequacy of ventilation), FENO (exhaled nitric oxide, for monitoring airway inflammation, pulmonary hypertension and cardiac failure) or other metabolic gases such as ketones in diabetic ketoacidosis, carbon monoxide, or core temperature. Attribute 44 may include changes in breathing cycle that, for example, may indicate hypopnea, and attribute 44 may indicate loss of pressure that may be indicative of apnea or a loose facemask.

As illustrated in FIG. 2, anti-asphyxiation valve 70 is received in regulator chamber 35 at arm end 34c of arm 33c to control inflow of ambient air 12 from ambient environment 97 into regulator chamber 35.

Anti-pathogen module 101 is received by arm 33b of regulator 30 in fluid communication with regulator chamber 35 of regulator 30 to remove pathogens from outflow gas 13, as illustrated. Pathogens, as used herein, may include, for example, pathogens such a viruses, bacteria, and fungi, as well as bodily fluids and various noxious, odiferous, or undesirable substances as may be included in outflow gas 13. Anti-pathogen module 101 may be omitted, in some implementations. Monitoring package 40 is secured to antipathogen module 101 in fluid communication with regulator chamber 35 to monitor attribute 44 of the outflow gas 13, as illustrated. Monitoring package 40 may be omitted, in some implementations. PEEP valve 90 is positioned downstream of monitoring package 40 in fluid communication with regulator chamber 35 of regulator 30 to maintain a selected baseline pressure p_{BL} within regulator chamber 35 as the user exhales, as illustrated. PEEP valve 90 may be omitted, in some implementations.

In the illustrated implementation, outflow gas 13 passes from regulator chamber 35 through anti-pathogen module 101, then through monitoring package 40, followed by passage through PEEP valve 90, and is discharged into the ambient environment from PEEP valve 90. Anti-pathogen module 101, monitoring package 40, and PEEP valve 90 may be arranged in other orders with respect to the flow of outflow gas 13, in various other implementations. Anti-pathogen module 101, monitoring package 40, and PEEP valve 90 are all optional, and, thus, may or may not be included, in various implementations.

Baseline pressure p_{BL} may be selected in order to maintain pressure on the most distal airways sufficient to prevents alveoli from collapsing during exhalation. Alveoli collapse may occur normally from absorption of oxygen in the alveolar sacs, and, unless these sacs are distended open, a ventilation perfusion mismatch and shunting develop resulting in loss of gas exchange ability. In ARDS (acute respiratory distress syndrome), loss of lung compliance may necessitate the use of PEEP valve 90 to improve oxygenation. PEEP valve 90 may be adjusted, for example, between 5-25 cm of water to set correspondingly the selected baseline pressure p_{BL}, as would be readily recognized by those of ordinary skill in the art upon study of this disclosure. PEEP valve 90 may be manufactured, for example, by Becton Dickinson and Company of Franklin Lakes, N.J., Ambu A/S of Denmark, or Besmed of New Taipei City, Taiwan.

As illustrated in FIG. 3, check valve 50a is disposed within arm passage 38a of arm 33a to control the flow of respiratory gas 11 through arm passage 38a into regulator chamber 35, and check valve 50b is disposed within arm passage 38b of arm 33b to control the flow of outflow gas 13 from regulator chamber 35 through arm passage 38b.

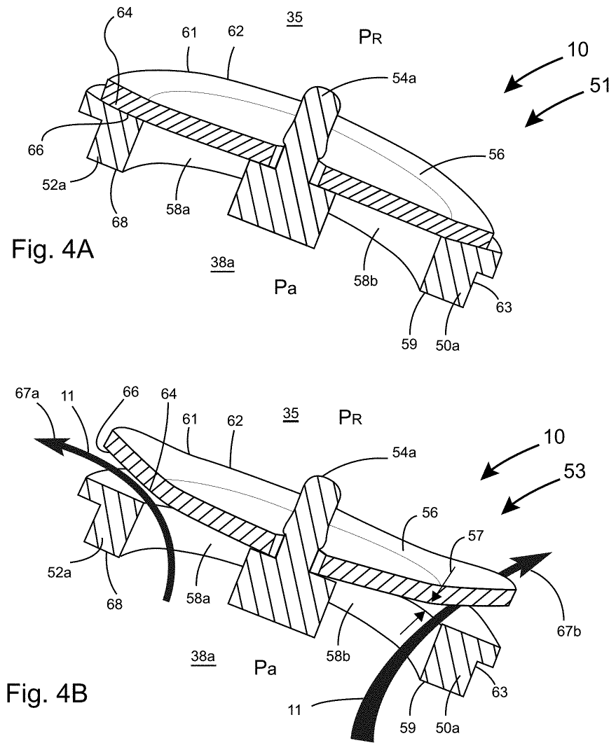

As illustrated in FIGS. 4A, 4B, check valve 50a includes valve member 56 insertably received over pin 54a that extends forth from valve seat 52a to engage valve member 56 with valve seat 52a. Check valve 50b, which includes pin 54b that extends forth from valve seat 52b (see FIG. 3), is formed similarly to check valve 50a and, thus, check valve 50b may operate similarly to check valve 50a, in this implementation. Valve seats 52a, 52b may be made of hard plastic, and the valve members of valves 50a, 50b, such as valve member 56, may be made of a soft, flexible material such as of rubber or silicone, in various implementations. Note that the valve members of valves 50a, 50b, such as valve member 56, are omitted from FIG. 3 for purposes of clarity of explanation, as are monitoring package 40, PEEP valve 90, and anti-pathogen module 101.

Valve seat 52a includes detent 63 formed around outer perimeter to engage with a corresponding detent (not shown) to secure check valve 50a to arm 33a within arm passage 38a, as illustrated. Apertures, such as apertures 58a, 58b, formed in valve seat 52a allow gas flow through valve seat 52a, in this implementation. As illustrated, check valve 50a is oriented so that surface 62 of valve member 56 is on the downstream side 61 of check valve 50a and surface 68 of valve seat 52a is on the upstream side 59 of check valve 50a, in this implementation. That is, pins 54a, 54b are oriented to extend forth from valve seats 52a, 52b, in a flow direction of respiratory gas 11 and outflow gas 13, respectively, in this implementation.

Check valve 50a is positionable between closed position 51 illustrated in FIG. 4A and open position 53 illustrated in FIG. 4B. In closed position illustrated in FIG. 4A, regulator pressure p_{R} within regulator chamber 35 on downstream side 61 of check valve 50a is greater than pressure pₐ within arm passage 38a on upstream side 59 of check valve 50a to hold portions of surface 64 of valve member 56 in biased engagement with portions of surface 66 of valve seat 52a. The biased sealing engagement of portions of surface 64 with portions of surface 66 sealingly engages valve member with valve seat 52a thus blocking gas flow through check valve 50a from downstream side 61 (e.g. regulator chamber 35) to upstream side 59 (e.g. arm passage 38a), in this implementation.

In open position 53 illustrated in FIG. 4B, pressure pₐ within arm passage 38a on upstream side 59 of check valve 50a is greater than regulator pressure p_{R} within regulator chamber 35 on downstream side 61 of check valve 50a to flex portions of surface 64 of valve member 56 in spaced relation with portions of surface 66 of valve seat 52a, in this implementation. When portions of surface 64 are in spaced relation with portions of surface 66 in open position 53, respiratory gas 11 may pass through check valve 50a from upstream side 59 (e.g., arm passage 38a) to downstream side 61 (e.g., regulator chamber 35) by passing through apertures, such as apertures 58a, 58b, in valve seat 52a and through gap 57 between portions of surface 64 of valve member 56 and portions of surface 66 of valve seat 52a, as indicated by arrows 67a, 67b in FIG. 4B.

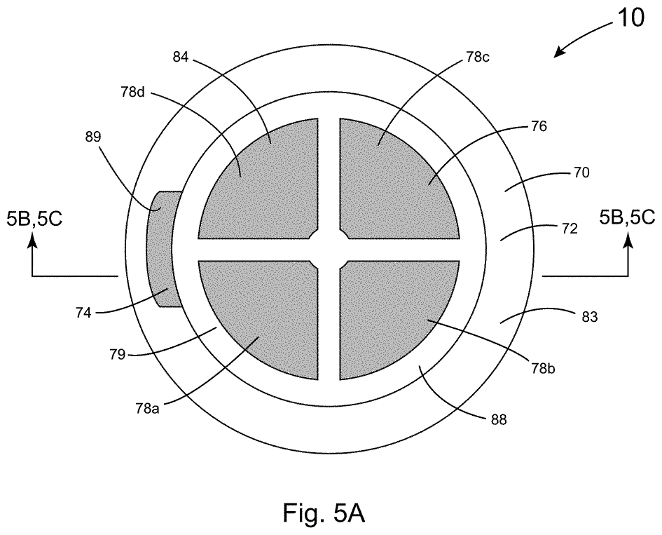

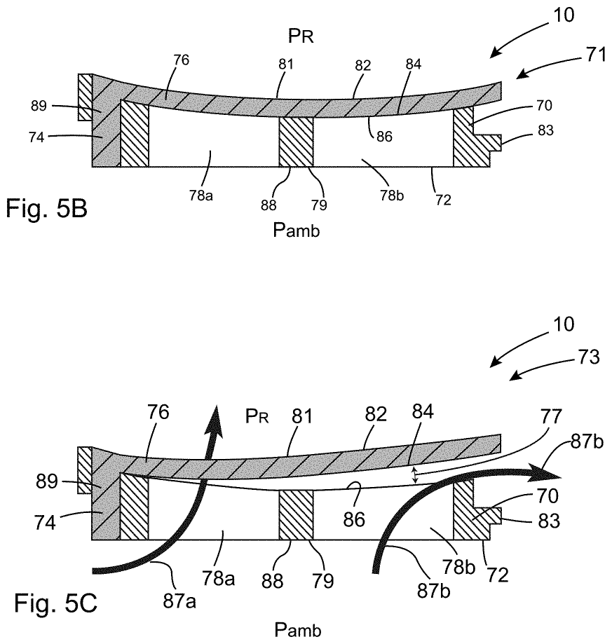

Anti-asphyxiation valve 70, which is illustrated, for example, in FIGS. 1B, 2, 3, 5A, 5B, 5C, is disposed within arm passage 38c of arm 33c to control the flow of ambient air 12 from the ambient environment 97 through arm passage 38c into regulator chamber 35. As illustrated in FIGS. 5A, 5B, 5C, anti-asphyxiation valve 70 includes valve member 76 secured to valve seat 72 by valve member arm 89 insertably securably received in arm detent 74 in periphery of valve seat 72. Valve member arm 89 which is unitary in structure with valve member 76, extends forth generally perpendicular to surface 84 of valve member 76 along a portion of the circumferential periphery of surface 84, in this implementation. Valve member 76 is thus cantilevered from valve member arm 89, in this implementation. Surface 86 of valve seat 72 may be slightly concave to enhance a cantilever action of valve member 76, in certain implementations. Valve seat 72 may be made of hard plastic, and valve member 76, may be made of a flexible material such as of rubber or silicone.

As illustrated, valve seat 72 is formed with detent 83 around at least portions of outer perimeter to engage with a corresponding detent (not shown) to secure anti-asphyxiation valve 70 to arm 33c within arm passage 38c, and arm detent 74 is formed in a portion of valve seat 72 proximate the outer perimeter of valve seat 72. Apertures 78a, 78b, 78c, 78d formed in valve seat 72 allow gas flow through valve seat 72, in this implementation. As illustrated, anti-asphyxiation valve 70 is oriented so that surface 82 of valve member 86 is on the downstream side 81 (e.g., regulator chamber 35) of anti-asphyxiation valve 70 and surface 88 of valve seat 72 is on the upstream side 79 (e.g., ambient environment 97) of anti-asphyxiation valve 70.

Anti-asphyxiation valve 70 is operably positionable between closed position 71 illustrated in FIG. 5B and open position 73 illustrated in FIG. 5C. In closed position 71, regulator pressure p_{R} within regulator chamber 35 on downstream side 81 of anti-asphyxiation valve 70 is greater than ambient pressure p_{amb} in ambient environment 97 on upstream side 79 of anti-asphyxiation valve 70 to hold portions of surface 84 of valve member 76 in biased engagement with portions of surface 86 of valve seat 72, as illustrated. In closed position 71, the biased sealing engagement of portions of surface 84 with portions of surface 86 sealingly engages valve member 76 with valve seat 72 thus blocking gas flow through anti-asphyxiation valve 70 from downstream side 81 to upstream side 79, in this implementation.

In open position 73 illustrated in FIG. 5C, ambient pressure p_{amb} on upstream side 79 of anti-asphyxiation valve 70 is greater than regulator pressure p_{R} within regulator chamber 35 on downstream side 81 of anti-asphyxiation valve 70 to flex portions of surface 84 of valve member 76 cantilevered from valve member arm 89 into spaced relation with portions of surface 86 of valve seat 72. When portions of surface 84 are in spaced relation with portions of surface 86 in open position 73, ambient air 12 may pass through anti-asphyxiation valve 70 from upstream side 79 (e.g. ambient environment 97) to downstream side 81 (e.g. regulator chamber 35) by passing through apertures 78a, 78b, 78c, 78d in valve seat 72 and through gap 77 between portions of surface 84 of valve member 76 and portions of surface 86 of valve seat 72, as indicated by arrows 87a, 87b, 87c in FIG. 5C.

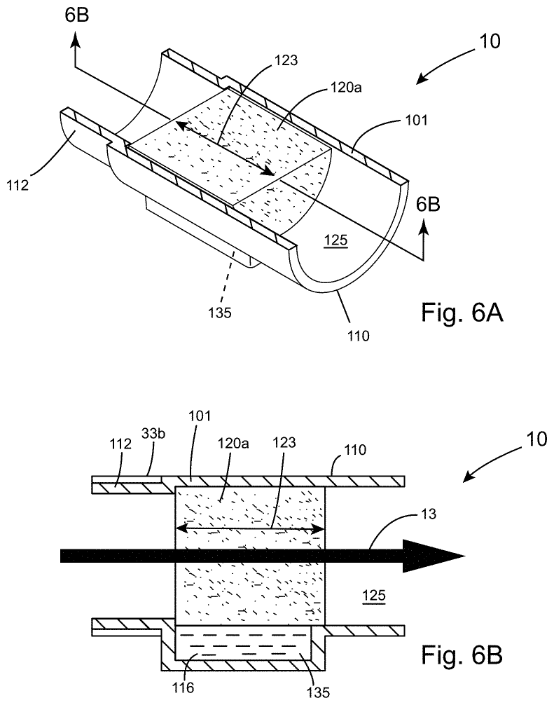

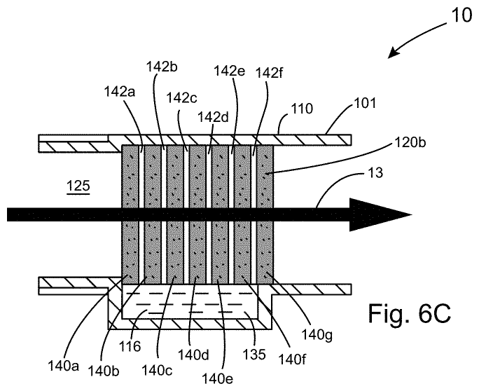

FIGS. 6A, 6B, 6C illustrate implementations of filter 120a, 12b of anti-pathogen module 101 that may optionally be included in respiratory support apparatus 10. Anti-pathogen module 101 is formed with a body 110 that is cylindrical with neck 112 designed to fit insertably securely within arm passage 38b at arm end 34b of arm 33b, as illustrated. Anti-pathogen module 101 defines cavity 125, and filter 120a is positioned within cavity 125, as illustrated in FIGS. 6A, 6B. Outflow fluid 13 passes through filter 120a, and filter 120a removes pathogens from outflow gas 13 prior to discharge of outflow gas 13 into the ambient environment, in this implementation.

As illustrated in FIGS. 6A, 6B, filter 120a is formed as a unitary structure. In various implementations, filter 120a may include any of a variety of available antimicrobial filters, for example, microporous hydrophobic membrane (such as those from Pall Filters) or melt blown polyethylene fibers. Filter 120a may include activated carbon, in various implementations. Filter 120a may include various combinations of materials, in various implementations. Length 123 of filter 120a within cavity 125 may be selected, for example, to conform anti-pathogen module 101 with HEPA standards.

In certain implementations, filter 120a may be treated with solution 116 to enhance pathogen removal from outflow gas 13 as outflow gas 13 passes through filter 120a. Solution 116 may have various anti-pathogenic properties and may be generally flowable. Solution 116 may include, for example, hydrogen peroxide. As illustrated, in FIG. 6B, solution 116 is stored in reservoir 135 in communication with filter 120a to flow onto filter 120a. Reservoir 135, which is defined by body 110, may have one or more apertures (not shown) between reservoir 135 and filter 120a sized to control communication of solution 116 from reservoir 135 onto filter 120a, for example, by capillary action, by diffusion, or by capillary action and diffusion. In other implementations, solution 116 may be applied directly to filter 120a. Because anti-pathogen module 101 is downstream from valve check valve 50b, the user may have little to no exposure to solution 116 including vapors that may emanate from solution 116.

Filter 120b that may be included in anti-pathogen module 101 in lieu of filter 120a is illustrated in FIG. 6C. In this exemplary implementation of FIG. 6C, filter 120b includes membranes 140a, 140b, 140c, 140d, 140e, 140f, 140g in spaced relation with one another to define gaps 142a, 142b, 142c, 142d, 142e, 142f therebetween, as illustrated in FIG. 6C. Other implementations may include more or fewer membranes and, thus, more or fewer gaps. Solution 116 may be communicated onto membranes 140a, 140b, 140c, 140d, 140e, 140f, 140g from reservoir 135. Gaps 142a, 142b, 142c, 142d, 142e, 142f may contain vapor from solution 116 that may enhance pathogen removal from outflow gas 13 as outflow gas 13 passes through gaps 142a, 142b, 142c, 142d, 142e, 142f. Anti-pathogen module 101 may, for example, include various combinations of filter 120a, 120b, in various implementations.

FIG. 7 illustrates monitoring package 40 in operable communication with arm passage 38b within arm 33b. Detector 41 of monitoring package communicates operably to detect attribute 44 of outflow gas 13 as outflow gas 13 passes through arm passage 38b. Locating detector 41 downstream of check valve 50b, especially when sampling is timed to peak expiratory flow or pressure may result in measurement of attribute 44 without dilution of attribute 44 by respiratory gas 11. Measurement and analysis of attribute 44 may yield useful information such as tidal volume calculated from a bell-shaped curve based on expiratory force over time, respiratory rate, core body temperature, etc. Changes in attribute 44 may alert the healthcare provider to changes in status of the user.

Detector 41 is in operable communication with controller 43 to allow controller 43 to control the detection of attribute 44 of outflow gas by detector 41 and to communicate data 42 indicative of attribute 44 of outflow gas 13 from detector 41 to controller 43. Communication interface 47 communicates with computer 49 via network 48. For example, controller 43 communicates with communication interface 47 to communicate data 42 indicative of attribute 44 of outflow gas 13 with computer 49 via communication interface 47. Computer 49, for example, may communicate with communication interface 47 and with controller 43 to control operations of communication interface 47, controller 43, and detector 41. FIG. 7 also includes sensor 29 that may communicate with computer 49 by network 23. In some implementations, sensor 29 may communicate via network 23 with communication interface 47 and then with computer 49 via network 48.

Controller 43 may include a microprocessor, clock, memory, A/D converter, and so forth, as would be readily recognized by those of ordinary skill in the art upon study of this disclosure. Communication interface 47 may be in wireless, wired, or both wireless and wired communication with computer 49 by network 48 in ways as would be readily recognized by those of ordinary skill in the art upon study of this disclosure. Monitoring package 40 communicates with a power supply (not shown) that may be mains electric or a battery that may be included in monitoring package 40. Monitoring package 40 may include a housing as well as various couplings, connectors, switches, interfaces for input or output, electrical pathways, and so forth, in various implementations, as would be readily recognized by those of ordinary skill in the art upon study of this disclosure.

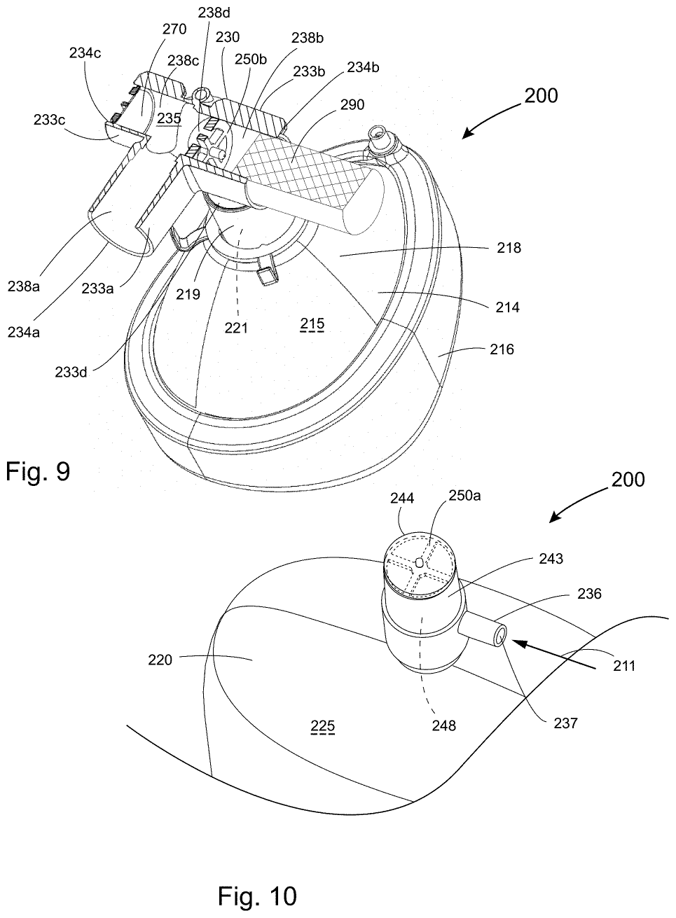

FIGS. 9, 10, 11 illustrate exemplary respiratory support apparatus 200 including regulator 230 secured to facemask 214. As illustrated, facemask 214 includes dome 218 surrounded peripherally by cushion 216, and mask conduit 219, which defines conduit passage 221, extends forth from dome 218. When secured to the user, facemask 214 defines facemask chamber 215 over the user's nose and mouth.

Regulator 230 includes arms 233a, 233b, 233c generally in coplanar disposition in the form of a “Y” or “T” and arm 233d generally normal to the coplanar disposition of arms 233a, 233b, 233c, in the illustrated implementation. Regulator 230 defines regulator chamber 235 and arms 233a, 233b, 233c, 233d define arm passages 238a, 238b, 238c, 238d, respectively, that communicate fluidly with regulator chamber 235, as illustrated in FIG. 9. Arm 233d may either insertably receive portions of mask conduit 219 within arm passage 238d or portions of arm 233d may be received within conduit passage 221 to secure regulator 230 to facemask 214 by interference fit with facemask chamber 215 in fluid communication with regulator chamber 235.

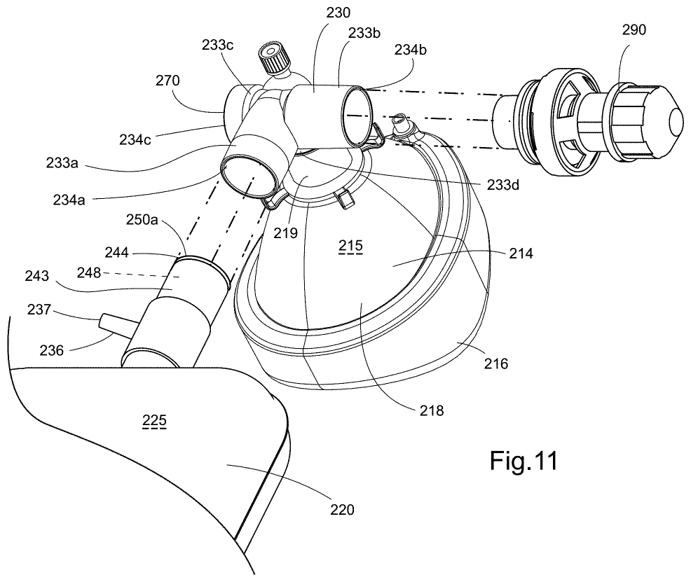

As illustrated in FIG. 10, bag 220 defines bag reservoir 225. Bag 220 is appended to bag conduit 243 that defines bag conduit passage 248 through which bag reservoir 225 fluidly communicates, as illustrated. Check valve 250a is received within bag conduit passage 248 proximate bag conduit end 244 opposite of bag 220, as illustrated in FIG. 10. Bag conduit end 244 and portions of bag conduit 243 may then be insertably received within arm passage 238a of arm 233a to position check valve 250a within arm passage 238a, as illustrated in FIG. 11. The portions of bag conduit 243 are held within arm passage 238a by interference fit. This contrasts with exemplary respiratory therapy apparatus 10 wherein check valve 50a is secured within passage 38a separable from bag conduit 39. When so received within arm passage 238a, check valve 250a controls fluid communication of respiratory gas 211 from inflow passage 237 and bag reservoir 225 with regulator chamber 235. Respiratory gas 211 may be communicated via inflow passage 237 into arm passage 238a and thence into regulator chamber 235 and/or into bag reservoir 225 of bag 220 as controlled by check valve 250a, as illustrated in FIGS. 9, 10. Thus, in exemplary respiratory support apparatus 200, check valves 250a, 250b and anti-asphyxiation valve 270 are disposed within arm passages 238a, 238b, 238c, respectively, to control fluid communications with regulator chamber 235 and, thus, with facemask chamber 215. As illustrated in FIG. 11, PEEP valve 290 may be insertably received within arm passage 238b at arm end 234b downstream of check valve 250b for securement by interference fit. PEEP valve 290 may further include a monitoring package and/or an anti-pathogen module, in various implementations. PEEP valve 290 may be omitted in certain implementations. As illustrated in FIG. 9, inflow port 236, which is formed as a nipple on arm 233a, defines inflow passage 237.

In exemplary operations of a respiratory support apparatus, such as respiratory support apparatus 10, 200, a facemask, such as facemask 14, 214, may be secured to a user to define a facemask chamber, such as facemask chamber 15, 215, over the user's nose and mouth so that the user inhales from the facemask chamber and exhales into the facemask chamber. A regulator, such as regulator 30, 230, may be secured to the facemask, and the regulator may include a PEEP valve, such as PEEP valve 90, 290, an anti-pathogen module, such as anti-pathogen module 101, and a monitoring package, such as monitoring package 40. The PEEP valve may be configured to set the selected baseline pressure p_{BL} within a regulator chamber, such as regulator chamber 35, 235, of the regulator, and, thus, within the facemask chamber and within the user's lungs 194 as the user exhales. The regulator may include a bag, such as bag 20, 220, that defines a bag reservoir, such as bag reservoir 25, 225. A respiratory gas, such as respiratory gas 11, 211 may be communicated with the regulator via an inflow port, such as inflow port 36, 236.

The facemask, the bag, and the PEEP valve may be provided as separate elements that may be joined together by interference fit, in various implementations. For example, a mask conduit, such as mask conduit 19, 219, may be engaged with an arm, such as arm 33d, 233d, by interference fit to secure the mask to the arm. A bag conduit, such as bag conduit 39, 243, may be engaged with an arm, such as arm 33a, 233a, by interference fit to secure the bag to the arm. The PEEP valve, monitoring package, and/or anti-pathogen module may be secured to an arm, such as arm 33b, 233b, by interference fit. Various guideways, keyways, stops, Luer lock fittings, and so forth may be provided that enable correct engagement of the mask conduit with the arm, the bag conduit with the arm, the PEEP valve with the arm, and gas source, such as gas source 99, in communication with the inflow port, in various implementations, as would be readily understood by those of ordinary skill in the art upon study of this disclosure.

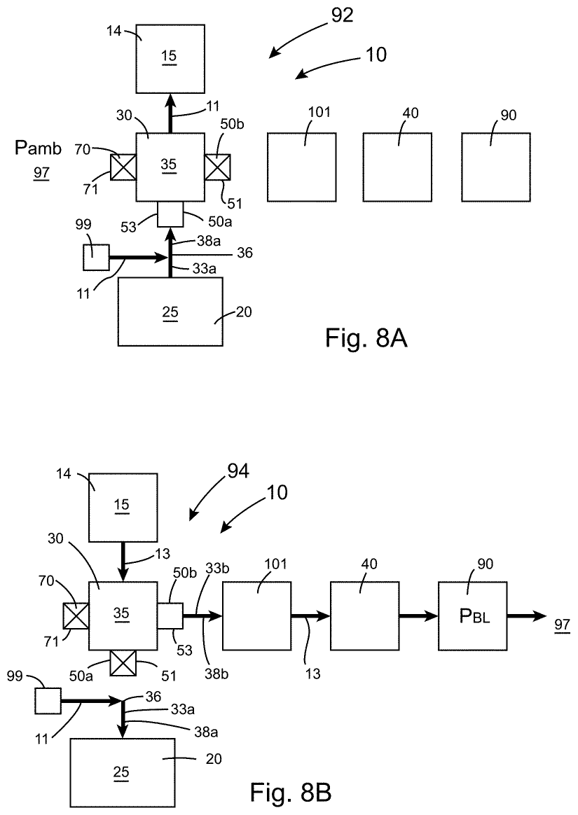

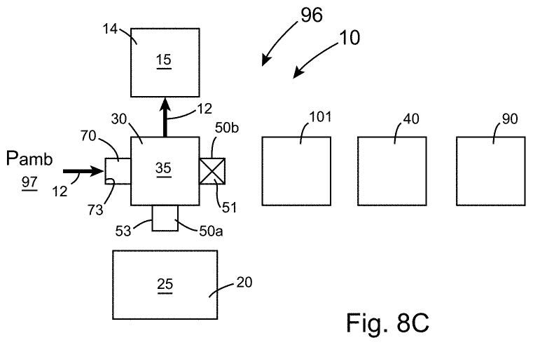

FIGS. 8A, 8B, 8C illustrate operations of respiratory support apparatus 10. Respiratory support apparatus 200 operates similarly to respiratory support apparatus 10. The respiratory gas 11 is flowed into arm passage 38a of arm 33a through inflow passage 37 of inflow port 36 from gas source 99, as illustrated in FIGS. 8A, 8B. Gas source 99 may be, for example, a cylinder of compressed gas or mains gas. In various implementations, gas source 99 may include an oxygen concentrator, such as an oxygen concentrator using zeolite molecular sieve. The oxygen concentrator may supply 85-94% oxygen as respiratory gas 11 at a continuous flow of 5 L/min (LPM), which is ample for the alveolar ventilation of a 70-Kg adult. An oxygen concentrator capable of 10 LPM continuous flow has been sourced and available for persons with higher tidal ventilation. In various implementations, gas source 99 may include an oxygen synthesizer such as an oxygen synthesizer that creates oxygen using electrolysis or fuel cell chemistry in combination with PEM (Proton Exchange Membrane). Gas source 99 may include a pressure regulator that allows regulating of pressure pₐ within arm passage 38a of arm 33a, for example, when check valve 50a is in closed position 51.

As illustrated in FIGS. 8A, 8B, 8C, exemplary respiratory support apparatus 10 may operate in exemplary first operational state 92, in exemplary second operational state 94, and in exemplary third operational state 96, respectively, as well as in states of operation intermediate of first operational state 92, second operational state 94, and third operational state 96. Respiratory support apparatus 10 transitions between first operational state 92, second operational state 94, and third operational state 96 as prompted by the user's spontaneous inhalation and exhalation, in this implementation. For example, as the user inhales, respiratory support apparatus 10 operates in first operational state 92 illustrated in FIG. 8A, and as the user exhales, respiratory support apparatus 10 operates in second operational state 94 illustrated in FIG. 8B. Third operational state 96, illustrated in FIG. 8C, prevents suffocation of the user from insufficient respiratory gas 11, or allows for controlled reductions of gas consumption from gas source 99. Note that, in the illustrated implementations of FIGS. 8A, 8B, 8C, check valves 50a, 50b are positioned between closed position 51 and open position 53 and anti-asphyxiation valve 70 is positioned between closed position 71 and open position 73 solely by the user's spontaneous breathing without assistance, for example, from electromechanical devices such as solenoid. Pressure differences between regulator pressure p_{R} within regulator chamber 35, pressure pₐ within arm passage 38a and pressure p_{b} within arm passage 38b position check valves 50a, 50b, respectively, between closed position 51 and open position 53, in various implementations. Pressure differences between regulator pressure p_{R} within regulator chamber 35 and ambient pressure p_{amb} in ambient environment 97 position anti-asphyxiation valve 70 between closed position 71 and open position 73, in various implementations. Thus, for example, no power source of electrical power is required to position check valves 50a, 50b, respectively, between closed position 51 and open position 53 and to position anti-asphyxiation valve 70 between closed position 71 and open position 73 thereby transitioning respiratory support apparatus 10 between first operational state 92, second operational state 94, and third operational state 96.

As the user inhales, respiratory support apparatus 10 operates in first operational state 92, as illustrated in FIG. 8A. As illustrated in FIG. 8A, respiratory gas 11 from gas source 99 flows into arm passage 38a of arm 33a via inflow port 36 and respiratory gas 11 flows into arm passage 38a of arm 33a from bag reservoir 25 of bag 20, in first operational state 92. Respiratory gas 11 is withdrawn from bag reservoir 25 into arm passage 38a during first operational state 94, thereby augmenting the flow of respiratory gas 11 from gas source 99 in order to provide sufficient respiratory gas 11 for inhalation by the user. Bag 20 is in expanded state 26 as first operational state 92 is initiated, and bag 20 is in collapsed state 22 when respiratory support apparatus 10 completes first operational state 92 due to withdrawal of respiratory gas 11 from bag reservoir 25 during first operational state 92.

As the user inhales, regulator pressure p_{R} within regulator chamber 35 decreases to less than pressure pₐ within arm passage 38a (e.g., pₐ>P_{R}) thereby placing check valve 50a in open position 53, and regulator pressure p_{R} within regulator chamber 35 decreases to less than pressure p_{b} within arm passage 38b (e.g., p_{b}>P_{R}) thereby placing check valve 50b in closed position 51. Check valve 50a in open position 53 allows respiratory gas 11 to flow from arm passage 38a through check valve 50a into regulator chamber 35 of regulator 30. Respiratory gas 11 then flows from regulator chamber 35 into mask chamber 15 of mask 14 for inhalation by the user.

Because check valve 50b is in closed position 51 in first operational state 92, there is no flow from regulator chamber 35 through check valve 50b into arm passage 38b of arm 33b. In first operational state 92, flow of respiratory gas 11 into regulator chamber 35 maintains regulator pressure p_{R} within regulator chamber 35 at greater than ambient pressure p_{amb} in ambient environment 97 (e.g., p_{R}>p_{amb}) to position anti-asphyxiation valve 70 is in closed position 71. Thus, there is no flow of ambient air 12 through anti-asphyxiation valve 70 into regulator chamber 35, in first operational state 92.

As the user exhales, respiratory support apparatus 10 operates in second operational state 94, as illustrated in FIG. 8B. In second operational state 94, regulator pressure p_{R} within regulator chamber 35 is greater than pressure p_{b} within arm passage 38b (e.g., p_{R}>P_{b}) due to user exhalation, thereby placing check valve 50b in open position 53, and regulator pressure p_{R} within regulator chamber is greater than pressure pₐ within arm passage 38a (e.g., p_{R}>pₐ), thereby placing check valve 50a in closed position 51, as illustrated.

With check valve 50b in open position 53, outflow gas 13, which comprises exhalation from the user flowing from mask chamber 15 into regulator chamber 35, flows from regulator chamber 35 through check valve 50b into arm passage 38b of arm 33b. Outflow gas 13 flows from arm passage 38b for discharge to ambient environment 97. As illustrated, outflow gas 13 flows successively from arm passage 38b through anti-pathogen module 101, through monitoring package 40, and through PEEP valve 90. Pathogens may be removed from outflow gas 13 by anti-pathogen module 101. Attribute 44 of outflow gas 13 may be detected by monitoring package 40, and the monitoring package may communicate data 42 indicative of attribute 44 to computer 49. Outflow gas 13 is discharged into ambient environment 97 from PEEP valve 90, as illustrated. Anti-pathogen module 101, monitoring package 40, and PEEP valve 90 may be disposed in various sequences so that outflow gas 13 may flow in various sequences through anti-pathogen module 101, monitoring package 40, and PEEP valve 90, in various other implementations. Any or all of anti-pathogen module 101, monitoring package 40, and PEEP valve 90 may be omitted, in various other implementations.

In second operational state 94, respiratory gas 11 flows into arm passage 38a of arm 33a and thence into bag reservoir 25 of bag 20 to replenish respiratory gas 11 within bag reservoir 25, as illustrated. Because check valve 50a is in closed position 51 in second operational state 94, there is no flow from arm passage 38a of arm 33a into regulator chamber 35. Bag 20, which may be in collapsed state 22 at the initiation of second operational state 94, may be in expanded state 26 at the completion of second operational state 94.

In second operational state 94, PEEP valve 90 maintains the regulator pressure p_{R} as greater than ambient pressure p_{amb} to place anti-asphyxiation valve 70 in closed position 71, in this implementation. Thus, as illustrated, there is no flow of ambient air 12 through anti-asphyxiation valve 70 from ambient environment 97 into regulator chamber 35 in second operational state 94. It should be noted that PEEP valve 90 sets baseline pressure p_{BL} within regulator chamber 35 during user exhalation that is greater than ambient pressure p_{amb}. For example, baseline pressure p_{BL} may be within a range of from about 5 mm H20 to about 25 mm H20. Regulator pressure p_{R} within regulator chamber 35 and pressures pₐ, p_{b} within arm passages 38a, 38b, respectively, may fluctuate with respect to baseline pressure p_{BL} and with respect to ambient pressure p_{amb} as the user inhales and exhales and check valves 50a, 50b are positioned between open position 51 and closed position 53.

In third operational state 96, the user inhales without sufficient respiratory gas 11 for the user to inhale an entirety of the user's tidal volume with the respiratory gas. Third operational state 96 may provide a safety measure that prevents suffocation of the user in the event the flow of respiratory gas 11 as per operational states 92, 94 is terminated, for example, due to human error or equipment failure. There is generally no flow of respiratory gas 11 from gas source 99 in third operational state 96, as illustrated in FIG. 8C, as indicated by check valve 50a in open position 53 without respiratory gas 11. Note that, in certain other implementations of third operational state 96, there may be inflow of respiratory gas 11 that is insufficient to enable the user to breathe therefore requiring supplementation with ambient air 12. In third operational state 96, regulator pressure p_{R} within regulator chamber 35 decreases to less than ambient pressure p_{amb} (e.g., p_{amb}>P_{R}) due to user inhalation thereby positioning anti-asphyxiation valve into open position 73, as illustrated. Anti-asphyxiation valve in open position 73 allows ambient air 12 to flow into regulator chamber 35 from ambient environment 97 and, thence, into mask chamber 15 of mask 14 for inhalation by the user, as illustrated in FIG. 8C. Check valve 50b is in closed position 51 to prevent ambient air 12 from flowing from regulator chamber 35 into arm passage 38b, respectively, during third operational state 96, as illustrated. Although check valve 50a is illustrated in open position 53, check valve 50a may be in closed position 51, or check valve 50a may fluctuate between open position 53 and closed position 51 during third operational state 96, in various implementations.

A combination of third operational state 96 with first operational state 92 may be entered at the end of first operational state 92 if a quantity of respiratory gas 11 is less than the lung capacity of the user. In such situations, the user draws respiratory gas into the lungs 194 (see FIG. 13) until the entire quantity of available respiratory gas 11 is drawn into the lungs 194. Continued inhalation then decreases regulator pressure p_{R} within regulator chamber 35 to less than ambient pressure p_{amb} thus positioning anti-asphyxiation valve 70 in open position 73 as in third operational state 96 thereby providing ambient air 12 to the user that may be in addition to respiratory gas 11 that may continue flowing through check valve 50a as in first operational state 92. The ambient air 12 may be inhaled proximate the end of an inhalation so that ambient air 12 fills respiratory pathways above the lungs 194 such as sinus cavities and bronchi and mask chamber 15 driving respiratory gas 11 deeper into the lungs 194. By being deeper in the lungs 194, oxygen as the respiratory gas 11 may be infused into the user while the anatomical dead space 196 (see FIG. 13) of the respiratory system that does not absorb oxygen is filled with ambient air 12.

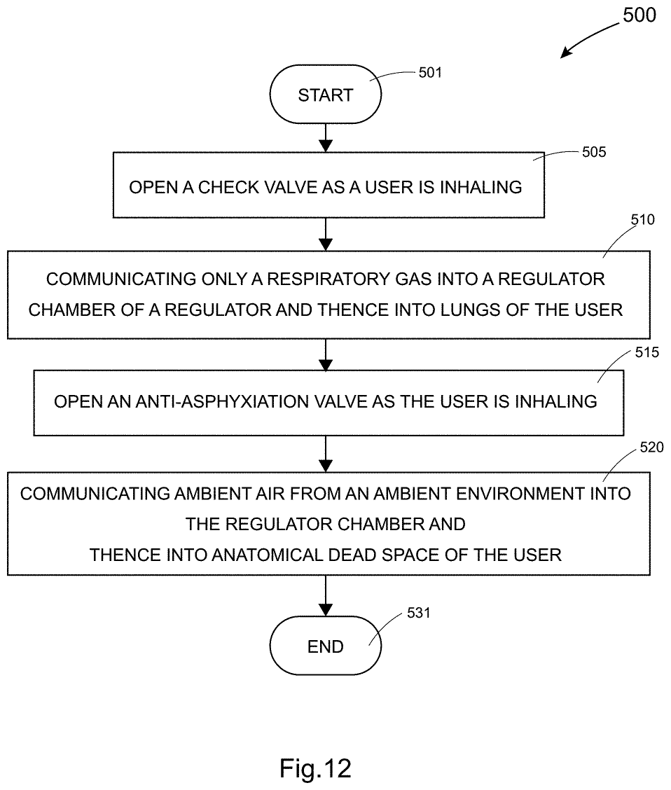

As an example of the combination of third operational state 96 with first operational state 92, the normal tidal volume (inspired breath) for a 70-kg man is about 500 ml. However, due to the approximately 150 ml anatomical dead space 196 including the oropharynx, nasopharynx, trachea and bronchi where no oxygen exchange takes place, as illustrated in FIG. 13, only 350 ml of the 500 ml actually reaches the alveoli where oxygen exchange occurs. If, for example, the user, such as user 199 of FIG. 13, inhales respiratory gas 11, the first 350 ml of respiratory gas 11 reaches the alveoli of the lungs 194, with the remaining 150 ml of respiratory gas 11 occupying the anatomical dead space 196. Additional respiratory gas 11 may also occupy facemask chamber 15 and regulator chamber 35 consuming an additional 75 ml to 100 ml or more of gas from gas source 99 without reaching lungs 194. The 150 ml of respiratory gas 11 in the anatomical dead space 196 along with respiratory gas 11 in facemask chamber 15 and regulator chamber 35 in this example is wasted because the respiratory gas 11 in the anatomical dead space 196, facemask chamber 15, and regulator chamber 35 provides no benefit to the user. Excluding the 150 ml from the 500 ml, it is only required to deliver 350 ml of respiratory gas to the lungs 194 per breath to support the user if the respiratory gas is configured to flow in first as the user inhales (see FIG. 12) with the remaining volume as the user inhales being supplied as ambient air 12.

For example, respiratory support apparatus 10 may be configured so that when the user inhales, the first 350 ml inhaled comprises respiratory gas 11 communicated into the lungs 194, and the remaining 150 ml inhaled comprises ambient air 12 communicated into the anatomical dead space 196. An additional volume of ambient air 12 may be delivered to facemask chamber 15 and regulator chamber 35. This, for example, conserves the 150 ml of respiratory gas 11 that would otherwise occupy the anatomical dead space 196 and may conserve an additional 100 ml respiratory gas 11 that would otherwise occupy at least portions of facemask chamber 15 and/or regulator chamber 35, resulting in about 35% to about 50% conservation of respiratory gas 11 that may be limited in supply. Thus, per this example, the available respiratory gas 11 is maximally used for alveolar oxygen exchange in lungs 194 and not wasted by placement in the anatomical dead space 196, facemask chamber 15, and regulator chamber 35 where no oxygen exchange takes place.

Respiratory support apparatus 10 may be used with an oxygen concentrator as gas source 99 in other than a hospital setting (e.g., a home or residential setting) and serve the acute and severe unmet needs of the affected masses, unable to enter the hospital care system. For example, a widely availableoxygen concentrator that supplies about 85% to about 94% oxygen at a 5 LPM continuous flow may provide the same high oxygenation clinically as a ventilator or HFNC to serve a 70-kg man.

As another example, respiratory support apparatus 10 may be useful in mountaineering where altitude sickness is common and yet the weight of supplies limits the amount of respiratory gas 11 that can be transported. Thus, reducing the quantity of respiratory gas 11 used per breath by 35% to 50% may reduce the size of gas source 99 transported by troops and climbers in high altitude situations. Reduction of supply weight is a high priority in high altitude missions. Conservation of the use of respiratory gas 11 may also be important in developing portions of the world such as Africa and parts of Asia where respiratory gas 11 may be a scare commodity.

This combination of third operational state 96 with first operational state 92 for respiratory support apparatus 10, 200 is illustrated in FIG. 12 as exemplary method 500. As illustrated in FIG. 12, exemplary method 500 is entered at step 501. At step 505, a check valve, such as check valve 50a, 250a, is opened as a user is inhaling.

As per step 510, opening the check valve at step 505 allows communication of only a respiratory gas, such as respiratory gas 11, 211, into a regulator chamber, such as regulator chamber 35, 235, of a regulator, such as regulator 30, 230. Only the respiratory gas is then communicated from the regulator chamber into lungs, such as lungs 194, of the user.

At step 515, an anti-asphyxiation valve, such as anti-asphyxiation valve 70, 270, is opened as the user is inhaling.

As per step 520, opening of the anti-asphyxiation valve at step 515 allows communication of ambient air, such as ambient air 12, into the regulator chamber and thence into anatomical dead space, such as anatomical dead space 196, of the user.

Exemplary method 500 terminates at step 531.

Steps 505, 510 are performed sequentially with steps 515, 520. First, at steps 505, 510, only the respiratory gas is communicated into the regulator chamber and thence into the lungs of the user. Then, at steps 515, 520, ambient air is communicated from the ambient environment into the regulator chamber and thence into the anatomical dead space of the user. The facemask chamber, such as facemask chamber 15, 215, of the facemask, such as facemask 14, 214, may also be filled, at least in part, with ambient air at the conclusion of steps 515, 520. The regulator chamber may also be filled, at least in part, with ambient air at the conclusion of steps 515, 520.

If it is desired to conserve the respiratory gas further or if the user does not need to inhale entirely respiratory gas, then the bag reservoir may be filled, for example, to only 200 ml or 100 ml as desired to provide the minimum oxygen enrichment or to conserve the oxygen supply. This may be suitable, for example, in incrementally weaning the user off of oxygen-enriched respiratory gas and returning the user towards breathing only ambient air.

In other implementations, the respiratory support apparatus may be used for increasing endurance of the user in reduced oxygen states (such as at high altitude) by increasing hemoglobin or red blood cell mass. The user may train initially wearing the apparatus without any oxygen supplementation, then switching to a progressively larger mask to increase the functional dead space upwards from 150 ml or higher until the target parameter is reached.

The mask and regulator add sufficient dead space to effectively reduce the amount of air reaching the alveoli for oxygen exchange. For example, reducing the amount of functional TV from 500 ml to 250 ml has the same effect as breathing room air with the oxygen concentration approximately halved. This may take place anytime anywhere. The training can be progressive in level, starting with only a small mask and progressing to a larger mask plus regulator—sans oxygen source. The end result is the enablement of mass training, easier enabled training with sustained results.

To counter a hypobaric environment, the respiratory support apparatus may include wi a compressor that pressurizes the respiratory gas such that the pressure provides a counter gradient to the hydrostatic pressure of the vascular system that is forcing plasma into the alveolar space thereby adversely affecting oxygen exchange. The presence of excessive plasma/interstitial fluid gives rise clinically to HAPE, a pulmonary edema-like state with diminished oxygen exchange. A continuous or intermittent positive airway pressure (PAP) may be deployed. The compression of respiratory gas may be provided, for example, by an electrically powered motor or even mechanically by the steps of the mountaineer pushing down on a bellow-like system that may be positioned beneath the foot. An adjustable valve may limit the degree of pressurization. The respiratory gas source may be worn beneath clothing to augment the compression, or if worn externally, may optionally be made of a deformation-resistant material to limit inflation.

PAP therapy patterns may include a boost of PAP that is timed to near the end of inhalation to help open up more alveoli, or as a battery saving measure, be used in conjunction with PEEP such that PAP is only needed during inhalation. Patterns may include pulsatile PAP that is timed to be delivered immediately at peak inhalation or immediately after systole in order to push more blood from the pulmonary system back to the heart, thus increasing venous return as the heart next relaxes during diastole.

The foregoing discussion along with the Figures discloses and describes various exemplary implementations. These implementations are not meant to limit the scope of coverage, but, instead, to assist in understanding the context of the language used in this specification and in the claims. The Abstract is presented to meet requirements of 37 C.F.R. § 1.72(b) only. Accordingly, the Abstract is not intended to identify key elements of the apparatus and methods disclosed herein or to delineate the scope thereof. Upon study of this disclosure and the exemplary implementations herein, one of ordinary skill in the art may readily recognize that various changes, modifications and variations can be made thereto without departing from the spirit and scope of the inventions as defined in the following claims.

1-20. (canceled)

21. A respiratory support apparatus, comprising:
a regulator having a regulator chamber defined at least in part by a first arm passage of a first arm, a second arm passage of a second arm, and a third arm passage of a third arm, the first arm being disposed at an angle with the third arm and the second arm being disposed at an angle with the third arm, the third arm being generally perpendicular to the first arm and to the second arm, and the third arm being attachable to a facemask conduit of a facemask for fluid communication between the regulator chamber and a facemask chamber of the facemask, the facemask adapted to cover an inspiratory aperture of a user with the mask conduit extending outward generally perpendicular from a face of the user;
a check valve received within the first arm passage of the first arm to control inflow of inflow gas into the regulator chamber; and
a second check valve received in the second arm passage of the second arm to control outflow of outflow gas from the regulator chamber to an ambient environment.

22. The apparatus of claim 21, wherein the first arm, the second arm, and the third arm are disposed in a T shaped configuration with the third arm forming a vertical portion of the T shaped configuration.

23. The apparatus of claim 21, further comprising:
an anti-asphyxiation valve in communication with the regulator chamber to allow ambient air to flow into the regulator chamber when the regulator pressure is less than ambient pressure.

24. The apparatus of claim 23, further comprising: a fourth arm perpendicular to the third arm with portions of a fourth arm passage of the fourth arm forming a portion of the regulator chamber, the anti-asphyxiation valve disposed in the fourth arm passage.

25. The apparatus of claim 21, further comprising:
a bag forming a bag reservoir in communication with the first arm passage of the first arm, the check valve controls at least in part exchange of inflow gas with the bag reservoir.

26. The apparatus of claim 25, wherein the bag is colored with a display color that facilitates observation of an expanded state of the bag, observation of a contracted state of the bag, and observation of transitions of the bag between the expanded state and the contracted state.

27. The apparatus of claim 21, further comprising:
a Positive End Expiratory Pressure valve (PEEP valve) downstream of the second check valve to set a baseline pressure within the regulator chamber.

28. The apparatus of claim 21, further comprising:
a filter positioned downstream of the second check valve to remove pathogens from the outflow gas.

29. The apparatus of claim 21, further comprising: a detector positioned downstream of the second check valve to detect an attribute of the outflow gas.

30. The apparatus of claim 29, wherein the attribute is selected from a group consisting of end-tidal CO₂ (EtCO2), ketones, nitric oxide, and temperature.

31. The apparatus of claim 30, wherein data related to the attribute is communicated to a computer.

32. The apparatus of claim 31, wherein the respiratory gas comprises oxygen at a concentration greater than that of ambient air.

33. A respiratory support apparatus, comprising:
a regulator having a regulator chamber defined at least in part by a first arm passage of a first arm, a second arm passage of a second arm, and a third arm passage of a third arm, the first arm and the second arm being generally coplanar and the third arm being generally perpendicular to the first arm and the second arm, and the third arm being attachable to a facemask conduit of a facemask for fluid communication between the regulator chamber and a facemask chamber of the facemask, the facemask adapted to cover an inspiratory aperture of a user with the mask conduit extending outward generally perpendicular to a face of the user;
an inflow port disposed on the first arm to communicate inflow gas into the first arm passage of the first arm;
a check valve received within the first arm passage of the first arm to control inflow of inflow gas into the regulator chamber, the check valve is actuated between a closed position and an open position by a pressure difference between a regulator pressure within the regulator chamber and a pressure on an opposing side of the check valve;
a bag forming a bag reservoir in communication with the first arm passage of the first arm, the check valve controls at least in part exchange of inflow gas between the first arm passage and the bag reservoir; and
a second check valve received in the second arm passage of the second arm to control outflow of outflow gas from the regulator chamber to an ambient environment, the second check valve is actuated between a second closed position and a second open position by a second pressure difference between the regulator pressure and a second pressure on a second opposing side of the second check valve, the second check valve is actuated between the second open position and the second closed position simultaneously as the check valve is actuated between the closed position and the open position.

34. The apparatus of claim 33, further comprising:
an anti-asphyxiation valve in communication with the regulator chamber to allow ambient air to flow into the regulator chamber when the regulator pressure is less than ambient pressure.

35. The apparatus of claim 34, further comprising: a fourth arm perpendicular to the third arm with portions of a fourth arm passage of the fourth arm forming a portion of the regulator chamber, the anti-asphyxiation valve disposed in the fourth arm passage.

36. The apparatus of claim 34, wherein the check valve is actuated into the open position and then the anti-asphyxiation valve is opened to communicate preferentially respiratory gas into lungs of the user and preferentially communicate ambient air into an anatomical dead space of the user.

37. The apparatus of claim 33, further comprising:
a Positive End Expiratory Pressure valve (PEEP valve) disposed about the second arm to set a baseline pressure within the regulator chamber.

38. The apparatus of claim 33, further comprising:
a filter positioned downstream of the second check valve to remove pathogens from the outflow gas.

39. The apparatus of claim 33, further comprising:
a detector positioned downstream of the second check valve to detect an attribute of the outflow gas

40. The apparatus of claim 39, wherein data related to the attribute is communicated via network to a computer.
